(19) United States
(12) Patent Application Publication
(10) Pub. No.: US 2008/0202506 A1
(43) Pub. Date: Aug. 28, 2008

(54) WATERFALL NEBULIZER

(76) Inventors: Derek D. Mahoney, Manalapan, NJ (US); George V. Muttathil, Cherry Hill, NJ (US); Kerry D. O'Mara, Hopewell Twp., NJ (US); Albert F. Stevens, Moorestown, NJ (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 12/038,505

(22) Filed: Feb. 27, 2008

Related U.S. Application Data
(60) Provisional application No. 60/891,892, filed on Feb. 27, 2007.

(51) Int. Cl. A61M 11/00

(52) U.S. Cl. 128/200.18; 128/200.21

(57) ABSTRACT

The present invention relates generally to a nebulizer, and more particularly but not exclusively to a compact nebulizer that efficiently utilizes medication.

### RELATED APPLICATIONS

This application claims the benefit of priority of U.S. Provisional Application No. 60/891,892 filed on Feb. 27, 2007, the entire contents of which application are incorporated herein by reference.

### FIELD OF THE INVENTION

The present invention relates generally to a nebulizer, and more particularly but not exclusively to a compact nebulizer that efficiently utilizes medication.

### BACKGROUND OF THE INVENTION

The deposition efficiency in the tracheobronchial (TB) and pulmonary regions is highly dependent on particle size. Particle sizes in the range of about 1 to 5 mm, as well as the size range extending from approximately 0.005 to 0.5 mm, have a relatively high rate of deposition within the aforementioned regions. Various methods have typically been used to generate these therapeutic fine particles, such as air-blast nebulizers (i.e., compressed air, jet, or venturi nebulizer), pressure nebulizers, ultrasonic nebulizers, a vibrating orifice, a spinning disk, condensation devices, and inkjet technology-based nebulizers. However, despite the variety of methods used to generate therapeutic fine particles, problems remain such as wasted medication that is not dispensed and the swallowing of liquid medication by the user. Currently available nebulizers typically have residual (i.e., waste) medication of 50% or more. Thus, it would be in advancing the state of nebulizer art to more efficiently dispense and utilize liquid medication, and to protect the user of the nebulizer from swallowing liquid medication.

### SUMMARY OF THE INVENTION

The present invention provides a nebulizer comprising or having a curved surface and a nozzle oriented so that outflow from the nozzle engages the curved surface. The nebulizer incorporates a nebulizer tube, which may comprise a single-piece, that may include a convergent-divergent, air mixing nozzle, as well as an integral feed channel for siphoning medication. The nebulizer tube independently provides a first-level (i.e., relatively coarse) atomization. To obtain the fine particles desired for nebulizers, the output stream from the nebulizer tube is directed towards an impactor having a curved surface at or proximate the impact site. When the flow strikes the impactor, very fine particles are generated. The curvature of the impactor promotes two very desirable effects. First, the portion of the flow that is not atomized into very fine particles will drain down the impactor and return to a medication reservoir disposed under the impactor, creating a “waterfall” recycling effect. Second, the impactor curvature also helps to direct the nebulized medication in a preferred direction, in this case toward the user's mouth. (The nebulizer of the present invention may be configured to substantially fit within the user's mouth, and this relatively small size of the nebulizer contributes to minimizing the amount of residual medication.)

The present invention also reduces the risk to the user associated with the inadvertent swallowing of unacceptably large quantities of liquid medication present in the nebulizer's reservoir. This could occur if the patient were to tilt his or her head too far back. To substantially reduce this risk, a semi-permeable membrane or other suitable material that is permeable to mist but sufficiently impermeable to liquid may be deployed to allow delivery of the nebulized mist to the user but prevent the flow of bulk liquid medication.

### BRIEF DESCRIPTION OF THE DRAWINGS

The foregoing summary and the following detailed description of the preferred embodiments of the present invention will be best understood when read in conjunction with the appended drawings, in which:

FIG. 1 schematically illustrates a perspective view of a first exemplary nebulizer of the present invention;

FIG. 2 schematically illustrates the nebulizer of FIG. 1, but without the semi-permeable membrane in place;

FIG. 3 schematically illustrates a cross-sectional view of the nebulizer of FIG. 2 taken along the sectioning line 3-3;

FIGS. 4A and 4B schematically illustrate perspective views of exemplary configurations of the lower housing of a nebulizer;

FIGS. 5A and 5B schematically illustrate perspective views of exemplary configurations of the lower housing of a nebulizer of the present invention having a enlarged region for receiving liquid medication;

FIGS. 6, 7A, and 7B schematically illustrate perspective views of exemplary configurations of the upper housing of the nebulizer of the present invention;

FIG. 8 schematically illustrates a cross-sectional view of a nebulizer similar to that depicted in FIG. 3, but including the lower housing of FIG. 4B and the upper housing of FIG. 7A;

FIG. 9 schematically illustrates the cross-sectional view of the nebulizer of FIG. 3 with the lower housing removed and with the upper housing rotated to show the internal cavity facing upward;

FIG. 10 schematically illustrates the perspective view of the nebulizer FIG. 2 with the lower housing removed and with the upper housing rotated to show the internal cavity facing upward;

FIGS. 11 and 12 schematically illustrate a perspective and cross-sectional view taken along the sectioning line 12-12, respectively, of a nebulizer tube of the present invention;

FIG. 13 schematically illustrates a perspective view of a second exemplary nebulizer of the present invention;

FIG. 14 schematically illustrates a cross-sectional view of the nebulizer of FIG. 13 taken along the sectioning line 14-14;

FIG. 15 schematically illustrates a perspective view of the lower housing of the nebulizer of FIG. 13;

FIG. 16 schematically illustrates a perspective view of the lower housing of the nebulizer of FIG. 13 with the nebulizer tube in place;

FIG. 17 schematically illustrates the nebulizer tube of FIG. 13 having key for insertion in the upper housing;

FIG. 18 schematically illustrates a perspective view of the upper housing of the nebulizer of FIG. 13 having a keyway for receiving the key of the nebulizer tube;

FIG. 19 schematically illustrates a perspective view of the upper housing of the nebulizer of FIG. 13 with the nebulizer tube in place with the key of the nebulizer tube disposed in the keyway of the upper housing;

FIGS. 20A and 20B schematically illustrate perspective views of a liquid fill cap; and

FIGS. 21A and 21B schematically illustrate alternative airfoil shapes for the impactor.

### DETAILED DESCRIPTION OF THE INVENTION

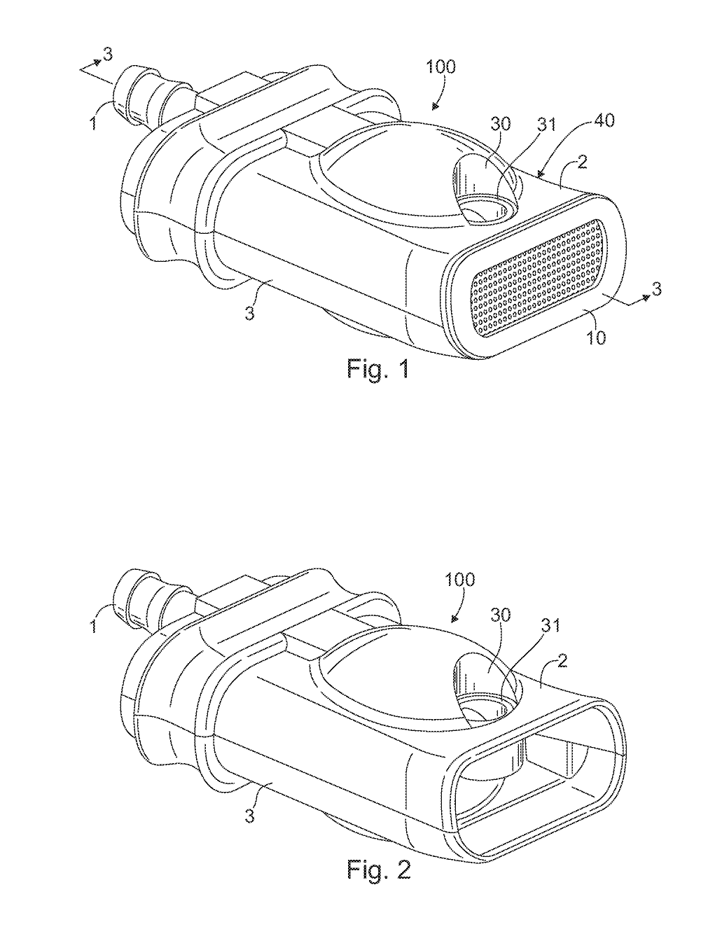

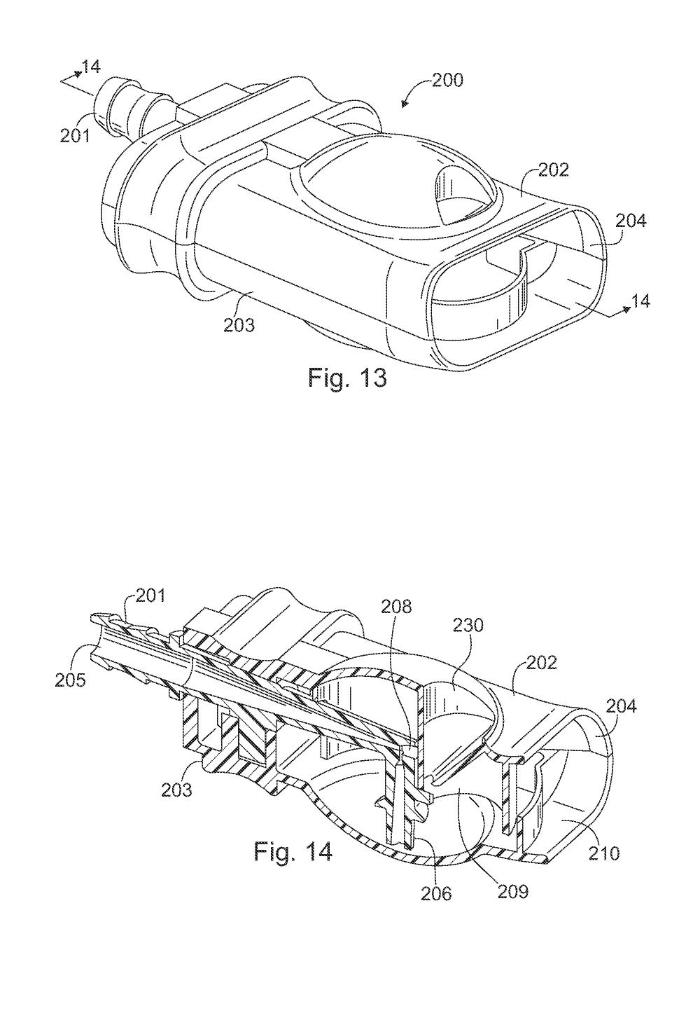

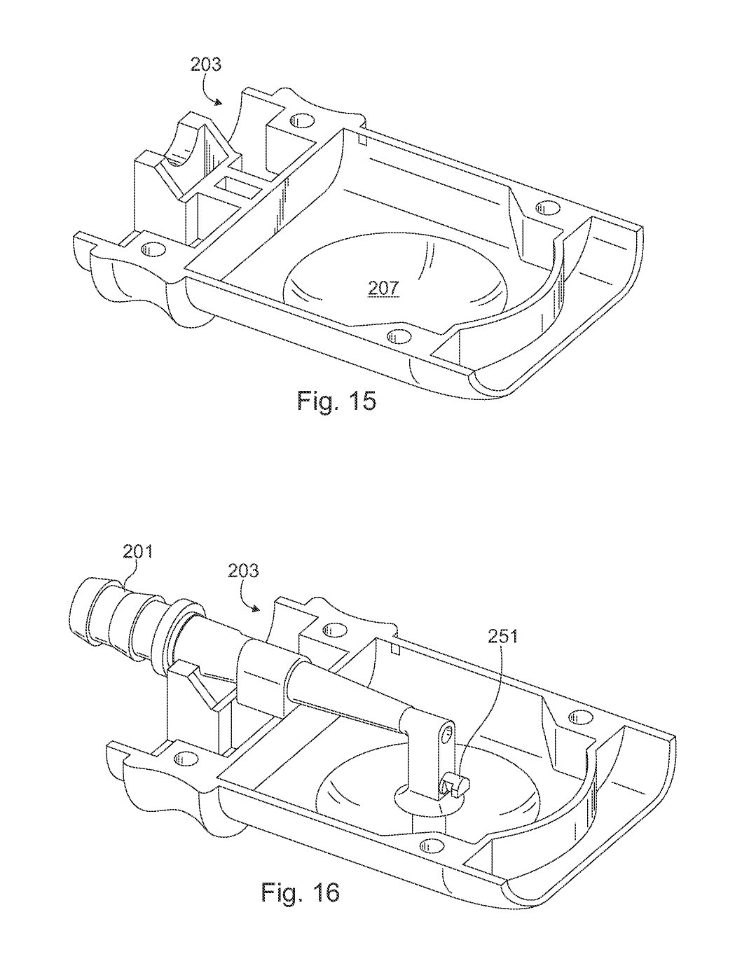

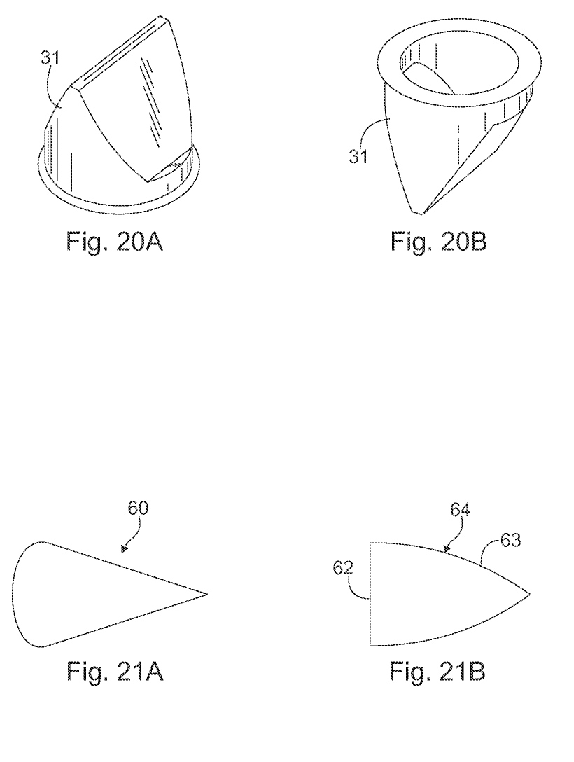

Referring now to the figures, wherein like elements are numbered alike throughout, FIGS. 1 and 2 illustrate an external view of a first configuration of a nebulizer 100 of the present invention. The nebulizer 100 comprises a nebulizer tube 1 disposed within a housing 40 for receiving compressed air and an exit port 10 for delivering a nebulized mist to a user. The housing 40 may comprise an upper housing 2 and a lower housing 3, which may be registered to one another by cooperation between holes 12 of the lower housing 3 and alignment posts 16 of the upper housing 2, FIGS. 4A, 6. The upper housing 2 may include a fill port 30 for introducing a liquid medication into the housing 40. The fill port 30 may be shaped to readily accept the shape of standard medicine containers, which will facilitate filling of the nebulizer 100 with the correct amount of medication and reduce the possibility of spillage and waste. The fill port 30 may remain open and may also serve as an exit for nebulized liquid, or the fill port 30 may optionally include a separate funnel or duckbill-shaped cap 31 for insertion into the upper housing 2 to direct the liquid medication into the housing 40, FIGS. 1, 3, 20A, 20B. Alternatively, the fill port cap 230 may be provided as an integral portion of the upper housing 202, FIGS. 13, 14. The cap 31, 230 may be configured so that it deflects to permit liquid to be poured into the nebulizer when a small force applied. For example, the cap 31, 230 may deflect when a syringe or tubular exit of a plastic ampule is inserted for delivering liquid and may close again after the syringe/ampule is removed.

To receive liquid medication introduced through the fill port 30, the lower housing 3 includes a reservoir 7 which may include a cylindrical sidewall 33 for containing the liquid medication within a localized region within the lower housing 3. The reservoir 7 may be dimensioned to hold at least 3 ml of liquid medication, for example. In addition, to further contain the location of the liquid medication, the reservoir 7 may include a hemispherical or other suitably shaped depression 34 into which the liquid medication may pool. Maintaining the liquid medication in a specified location assists in making the medication available to the nebulizer tube 1, and thus aids in efficient use of the medication.

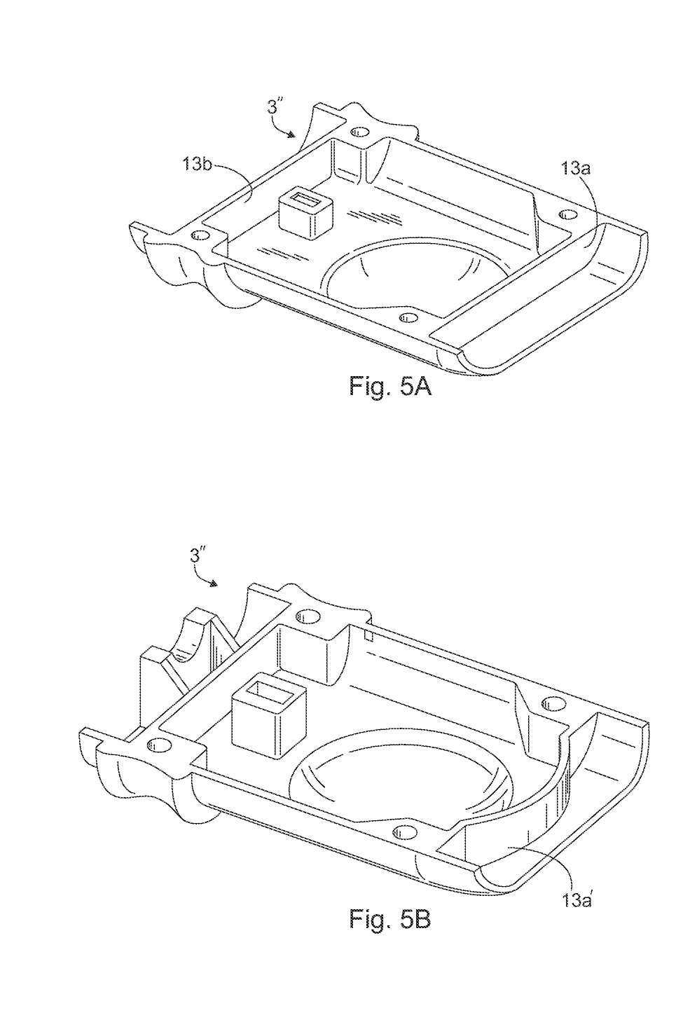

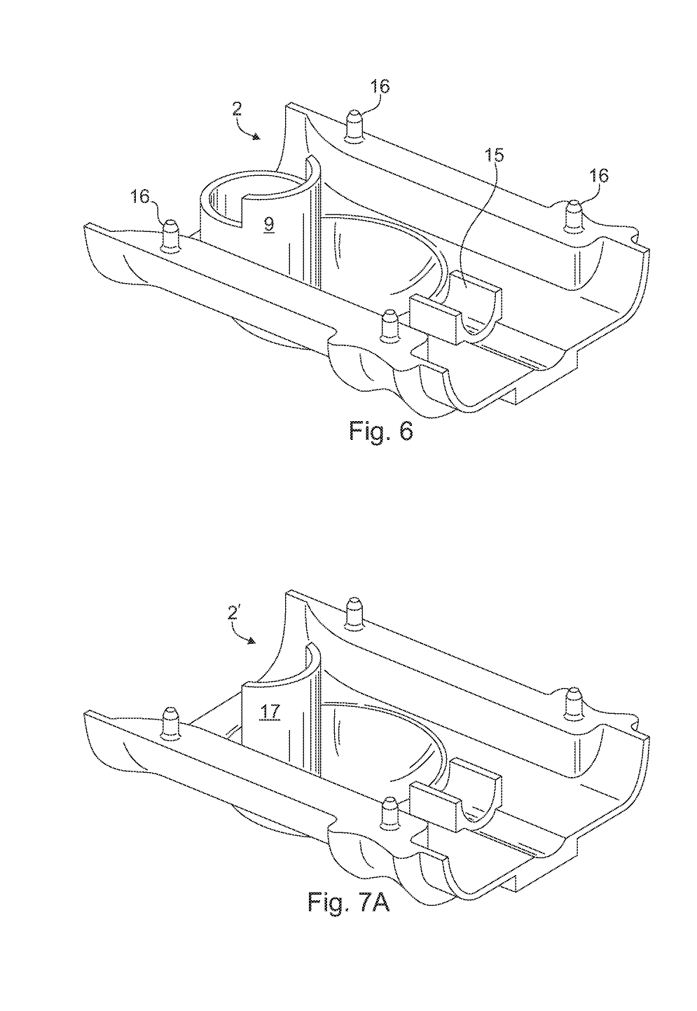

The reservoir 7 may include shapes other than cylindrical. For example, the reservoir 7″ may have a generally rectangular shape being bounded at the inlet and outlet end of the lower housing 3″ by front and rear reservoir walls 13a, 13b, FIG. 5A. The reservoir walls 13a, 13b may be straight, curved 13a′, or assume any other suitable shape, FIGS. 5B, 16. In addition, in the event that liquid medication overflows the wall 33′ of the reservoir 7′, an overflow wall 13 may optionally be provided at the exit port 10 to help deter introduction of liquid medication into the user's mouth, FIG. 4B. Furthermore, one or more semi-permeable membranes 4 may be provided at the exit port 10 of the nebulizer 100 to permit vapor flow while acting as an effective liquid barrier, thus creating a safety feature that prevents a user from swallowing liquid medication contained in the nebulizer 100. In one configuration the semi-permeable membranes 4 may be used instead of the front reservoir wall 13a. Alternatively, or additionally, an absorbent material, such as a sponge, may be incorporated into the nebulizer 100, for example between the reservoir 7′ and overflow wall 13, to deter the outflow of liquid medication into the exit port 10. For instance, in the event that the nebulizer is tilted beyond some critical angle during use, the membrane 4 and/or absorbent material will block the flow of medication into the user's mouth.

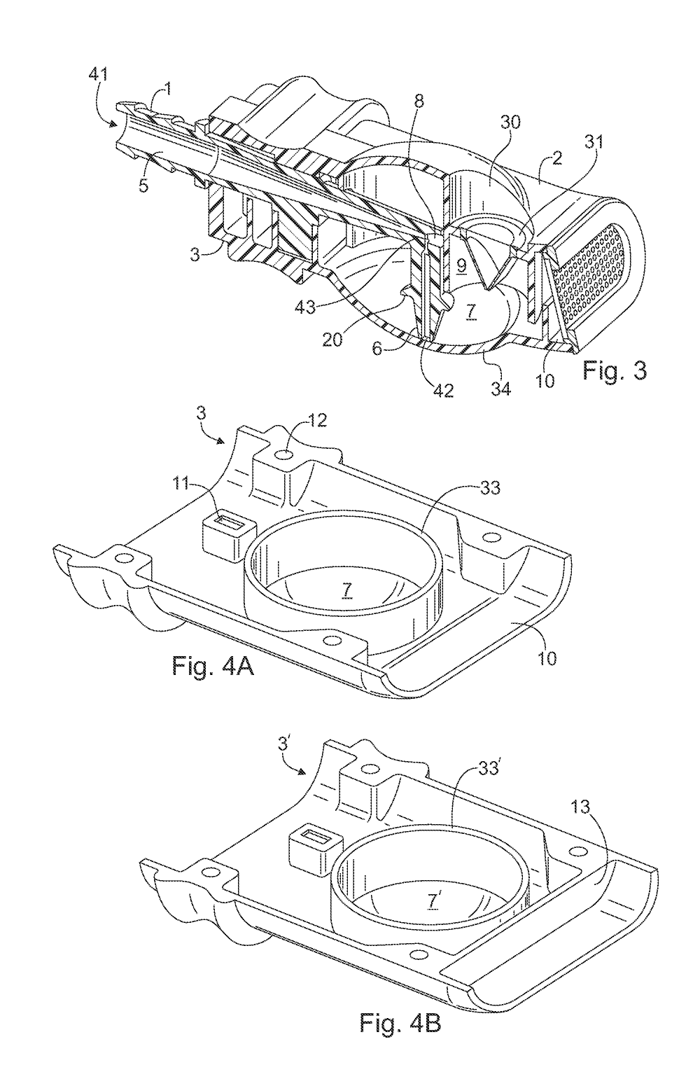

The nebulizer tube 1 includes a feed channel 6 having an inlet end 42 disposed in fluid communication with the reservoir 7 to receive liquid medication disposed within the lower housing 3, FIGS. 3, 12. The feed channel 6 communicates with an air channel 5 of the nebulizer tube 1 to deliver the liquid medication to the air channel 5 to be nebulized. The air channel 5 includes an inlet end 41 for connection to a source of compressed air and a throat 43 where the feed channel 6 connects to the air channel 5. The air channel 5 may be provided in the form of a convergent channel 5 that has a cross-sectional dimension that decreases from the inlet end 41 to the throat 43 where the cross-sectional dimension may be a minimum. The nebulizer tube 1 also includes a nozzle 8 disposed in fluid communication with the throat 43 of the air channel 5. The nozzle 8 include a channel cross-sectional dimension that increases away from the throat 43 towards the outlet end 44 of the nebulizer tube 1.

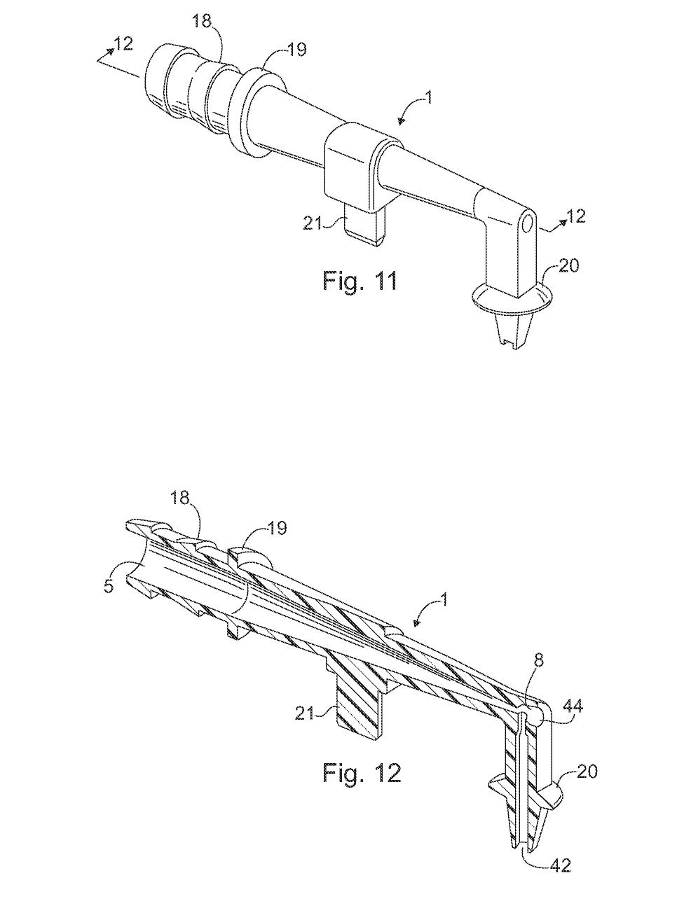

The inlet end 41 of the nebulizer tube 1 may include a barb 18 to assist in securing attachment of a compressed air hose to the inlet end 41 of the nebulizer tube 1, FIGS. 11, 12. A flange 19 may also be included to provide a positive stop for the air hose during initial installation. During operation, compressed air enters the convergent channel 5 of the nebulizer tube 1. The air accelerates until it reaches the throat 43 of the convergent channel 5. By virtue of the Bernoulli effect, as the flow velocity increases, its static pressure will decrease. As a result, the pressure at the throat 43 of the convergent channel 5 will be below that of the local atmospheric pressure. Since the static pressure of the liquid is higher than the pressure at the throat 43 of the nebulizer tube 1, liquid is siphoned upward into the feed channel 6 as a result of a venturi effect. Subsequent to siphoning, the liquid/air mixture is rapidly expanded in the divergent section of the nozzle 8. This rapid expansion encourages turbulent mixing and creates an effective first-level of atomization.

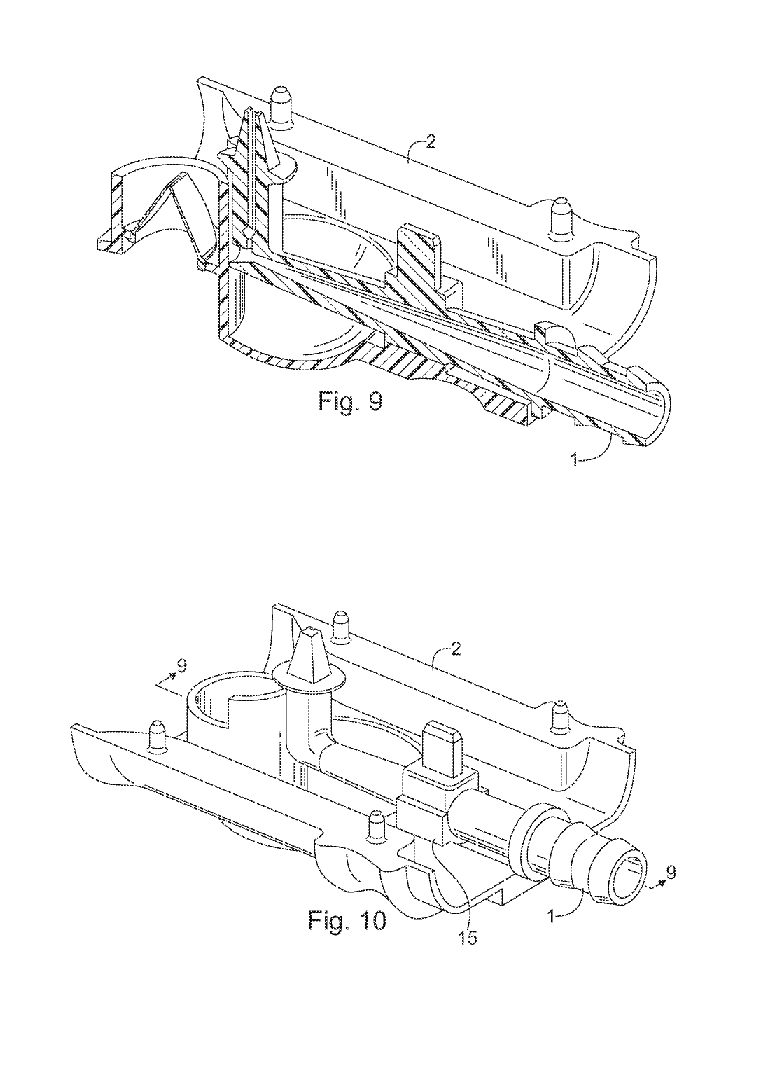

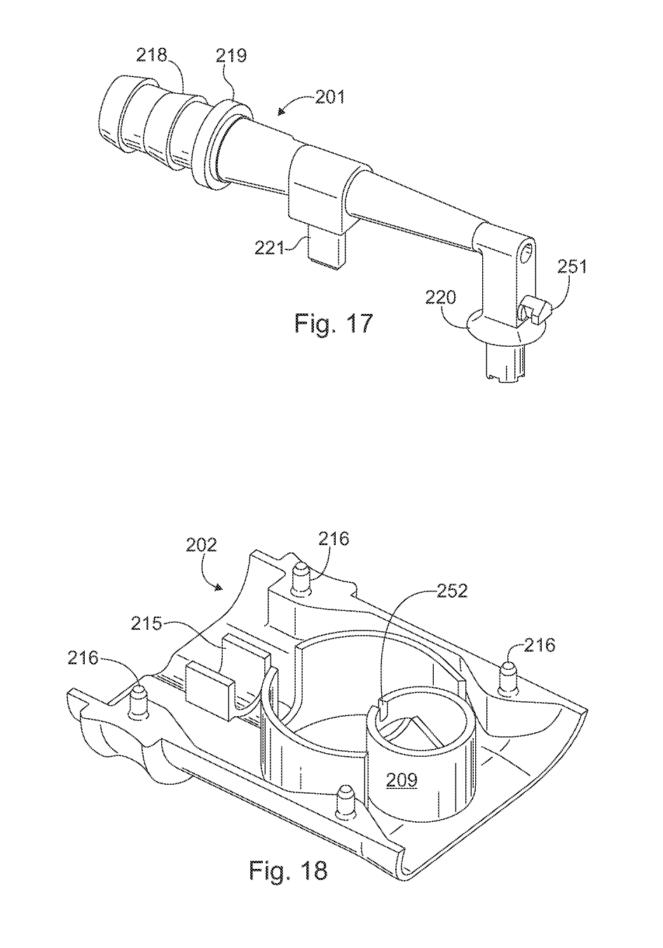

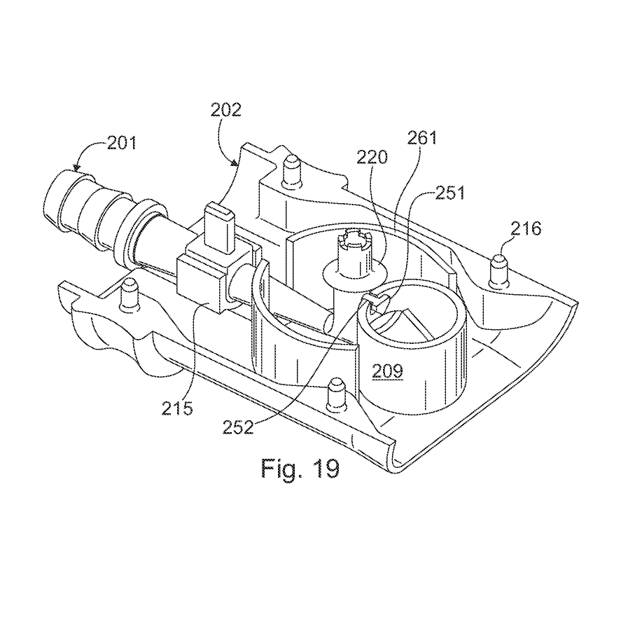

The nozzle 8 is oriented so that the output flow from the nozzle 8 strikes a curved impactor 9, which may be provided as part of the upper housing 2. This energetic collision generates the very fine, therapeutic particles required of nebulizers. It has been determined that a sufficiently small spacing is required between the nozzle 8 and impactor for fine mist generation. A suitable nozzle to impactor spacing is 10 or 30 thousandths of an inch. The location of the nozzle 8 relative to the curved impactor 9 may be specified by an alignment boss 21 provided on the nebulizer tube 1 that mates with a complementary positioning feature 11 of the lower housing 3 to locate the nebulizer tube 1 within the housing 40. In addition, the nebulizer tube mates with an nozzle capture feature 15 of the upper housing 2 to stabilize the tube 1 within the nebulizer 100, FIGS. 8-10. Additionally, or alternatively, registration of the nebulizer tube 1 to the impactor 9 may be provided by direct or indirect physical cooperation between the nebulizer tube 1 and impactor 9. For example, referring to FIGS. 16-19 (wherein structures similar to those illustrated in FIGS. 1-12 are similarly numbered with a “200”-series reference numeral), the nebulizer tube 201 may include a registration feature, such as a key 251, for mating with a complementary structure, such as keyway 252, on the nebulizer 209. Engagement between the key 251 and the keyway 252 establishes the relative position between the nozzle 208 and the impactor 209.

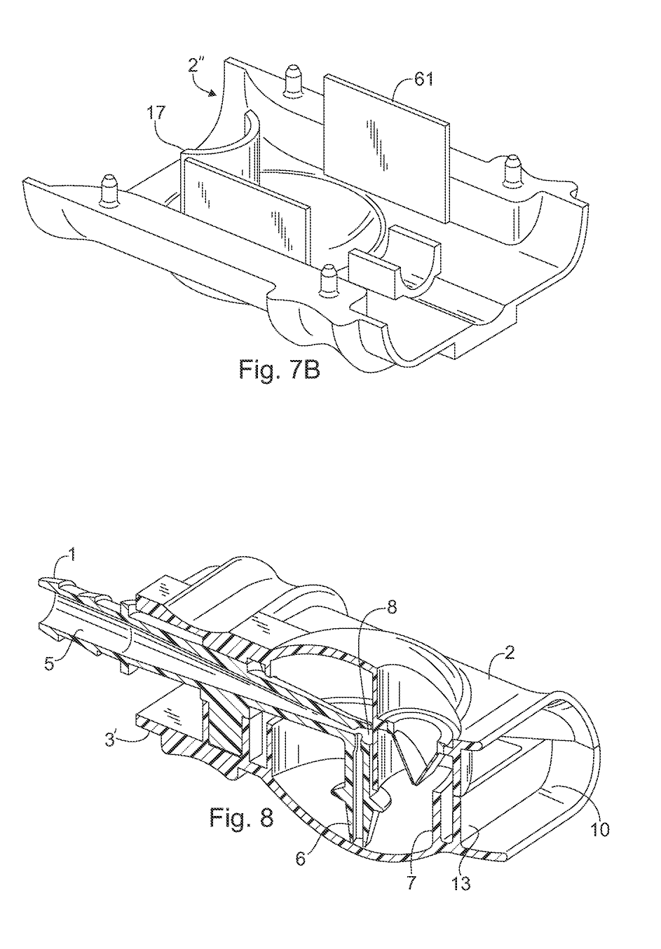

The impactor 9, 209 may have a generally cylindrical shape, such as a substantially full cylinder, FIG. 6, or a partial cylindrical impactor 17, FIG. 7A. Such impactor shapes will generate a fine mist and will also facilitate the flow of mist toward the user's mouth. Other curved surfaces may be substituted for the cylindrical impactors 9, 209 such as elliptical, or other suitable shape, e.g., an airfoil 60, FIG. 21A. In addition, the curved impactor may have a cross-sectional shape which includes a flat region 62 as well as a curved region 63, such as the airfoil 64 illustrated in FIG. 21B, for example. The airfoil impactor 60, 64 is oriented within the housing 40, 240 so that the tapered portion of the airfoil points in the downstream direction towards the exit port 10, 210 of the nebulizer 100, 200. Such an orientation of the airfoil impactor 60, 64 would reduce turbulence and backpressure of the air and mist as it moves out the exit port 10, 210 of the nebulizer 100, 200.

In addition to creating a fine mist, the curved impactor 9 also provides at least two other desirable functions: (I) it helps direct the nebulized mist towards the user's mouth, and (ii) it facilitates a waterfall-like recycling effect. The waterfall effect arises because part of the mixture exiting the nebulizer tube 1 will strike the impactor 9 and simply drain back down into the region containing the pool of liquid, i.e., reservoir 7. In this regard, the impactor 9 may be positioned above the reservoir 7. Of course, a significant portion of the air/liquid mixture will exit via port 10 of the nebulizer as a mist directed toward the user's mouth. An air baffle 20 may be provided on the nebulizer tube 1 proximate the feed channel inlet end 42, so that the high-velocity mixture striking the impactor 9 does not blow liquid away from the feed channel inlet 42 which could lead to a feed channel starvation condition. In addition, inclusion of the air baffle 20 can deter unwanted formation of large airborne droplets that might result from the surface of the liquid being agitated.

Additionally, the impactor 9, 209 can be shaped to create a scavenging flow within the nebulizer 100, 200. The scavenging flow would be directed throughout the housing interior and would help prevent the accumulation of medication on the internal walls of the nebulizer 100, 200. In addition, curtain walls 61, 261 are provided in the upper housing 2, 202 to redirect any accumulation of liquid on the upper surface of the upper housing 2, 202 downward into the reservoir 7, 207. The presence of curtain walls 61, 261 can avoid the situation of liquid running down the interior sidewall of the upper housing 2,202 to encounter and potentially leak out through the seam between the upper housing 2, 202 and the lower housing 3, 203. The curtain walls 261 are also positioned sufficiently close to the impactor 209 to permit fine particles to travel around the impactor 209 to the exit port 210 and to cause larger particles to strike the curtain walls 261 and then drip down into the reservoir 207. Additionally, a filter-type material may be positioned in the nebulizer 100, 200 to give a preferential flow direction for the nebulized mist toward the user's mouth without creating an excessive flow resistance to inhalation. Furthermore, the housing 40, 240 and/or other components of the nebulizer 100, 200 may be fabricated from materials that possess surface tension properties characteristic of wetting materials to create a sheeting action that will facilitate the flow of recycled materials to the reservoir 7, 207.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as set forth in the claims.

What is claimed is:

1. A nebulizer for delivering a mist of liquid, comprising:
a housing having an exit port for delivering a mist of liquid;
a reservoir disposed internally to the housing for containing a liquid to be nebulized;
a nebulizer tube having:
a gas channel having a first end for receiving a compressed gas and a second end for expelling compressed gas and nebulized liquid, and
a liquid feed channel having a first end in fluid communication with the reservoir for receiving the liquid from the reservoir and having a second end in fluid communication with the gas channel, whereby application of compressed gas to the first end of the gas channel creates a siphon in the liquid feed channel to draw liquid into the feed channel and to expel the liquid and compressed gas from the second end of the gas channel; and
an impactor disposed proximate the second end of the gas channel to nebulize the expelled liquid when the expelled liquid strikes the impactor, the impactor having a curved surface to direct the nebulized liquid towards the housing exit port.

2. The nebulizer according to claim 1, wherein the second end of the nebulizer tube is disposed proximate the curved surface of the impactor so that the expelled liquid strikes the curved surface of the impactor.

3. The nebulizer according to claim 1, wherein the impactor has an airfoil cross-sectional shape.

4. The nebulizer according to claim 1, wherein the impactor includes a tapered portion pointing in a downstream direction towards the exit port of the nebulizer.

5. The nebulizer according to claim 1, wherein the impactor includes a generally cylindrical or elliptical shape.

6. The nebulizer according to claim 1, wherein the impactor has a flat surface, and the second end of the gas channel is disposed proximate the flat surface of the impactor so that the expelled liquid strikes the flat surface of the impactor.

7. The nebulizer according to claim 1, wherein the second end of the gas channel comprises a convergent to divergent passageway to provide a nozzle to enhance atomization.

8. The nebulizer according to claim 1, comprising a membrane disposed at the exit port of the nebulizer to allow vapor flow and deter the passage of liquid therethrough.

9. The nebulizer according to claim 8, wherein the membrane is a semi-permeable membrane.

10. The nebulizer according to claim 1, comprising an absorbent material proximate the exit port of the nebulizer to deter the passage of liquid through the exit port.

11. The nebulizer according to claim 1, comprising an overflow wall disposed between the reservoir and the exit port to deter the passage of liquid through the exit port.

12. The nebulizer according to claim 1, wherein the housing comprises an upper housing portion and a lower housing portion each housing portion having a sidewall with an interior wall surface, wherein the upper and lower housings may be joined together and wherein the upper housing comprises a curtain wall that extends downward into the lower housing portion past the point of joinder between the upper and lower housing portions.

13. The nebulizer according to claim 1, wherein the housing comprises an upper housing portion and a lower housing portion that may be joined together to form a enclosure, each housing portion having a sidewall with an interior wall surface, and wherein the nebulizer comprises a curtain wall that is positioned to provide a protective curtain at the point of joinder between the upper and lower housing portions to deter the passage of liquid into the point of joinder.

14. The nebulizer according to claim 13, wherein the curtain wall is configured to redirect any accumulation of liquid on the upper surface of the upper housing portion downward into the reservoir.

15. The nebulizer according to claim 14, wherein the curtain wall is positioned relative to the impactor to permit the mist to travel by the curtain wall around the impactor to the exit port.

16. The nebulizer according to claim 13, wherein the curtain wall comprises a curved surface.

17. The nebulizer according to claim 1, wherein the reservoir comprises depression into which the liquid may pool and wherein the first end of the liquid feed channel is disposed proximate the deepest portion of the reservoir.

18. The nebulizer according to claim 1, wherein the gas channel comprises a convergent channel that has a cross-sectional dimension that decreases from the first end toward the second end and has a minimal cross-sectional dimension proximate the second end of the liquid feed channel.

19. A nebulizer for delivering a mist of liquid, comprising:
a housing having an exit port for delivering a mist of liquid;
a reservoir disposed internally to the housing for containing a liquid to be nebulized;
a monolithic nebulizer tube having:
a gas channel having a first end for receiving a compressed gas and a second end for expelling compressed gas and nebulized liquid, and
a liquid feed channel having a first end in fluid communication with the reservoir for receiving the liquid from the reservoir and having a second end in fluid communication with the gas channel, whereby application of compressed gas to the first end of the gas channel creates a siphon in the liquid feed channel to draw liquid into the feed channel and to expel the liquid and compressed gas from the second end of the gas channel; and
an impactor disposed proximate the second end of the gas channel to nebulize the expelled liquid when the expelled liquid strikes the impactor.

20. A nebulizer for delivering a mist of liquid, comprising:
a housing having an exit port for delivering a mist of liquid;
a reservoir disposed internally to the housing for containing a liquid to be nebulized;
a nebulizer tube having:
a gas channel having a first end for receiving a compressed gas and a second end for expelling compressed gas and nebulized liquid, and
a liquid feed channel having a first end in fluid communication with the reservoir for receiving the liquid from the reservoir and having a second end in fluid communication with the gas channel, whereby application of compressed gas to the first end of the gas channel creates a siphon in the liquid feed channel to draw liquid into the feed channel and to expel the liquid and compressed gas from the second end of the gas channel, wherein the second end of the gas channel comprises convergent to divergent passageway to provide a nozzle to enhance atomization; and
an impactor disposed proximate the second end of the gas channel to nebulize the expelled liquid when the expelled liquid strikes the impactor.

21. A nebulizer for delivering a mist of liquid, comprising:
a housing having an exit port for delivering a mist of liquid;
a reservoir disposed internally to the housing for containing a liquid to be nebulized;
a nebulizer tube having:
a gas channel having a first end for receiving a compressed gas and a second end for expelling compressed gas and nebulized liquid, and
a liquid feed channel having a first end in fluid communication with the reservoir for receiving the liquid from the reservoir and having a second end in fluid communication with the gas channel, whereby application of compressed gas to the first end of the gas channel creates a siphon in the liquid feed channel to draw liquid into the feed channel and to expel the liquid and compressed gas from the second end of the gas channel;
an impactor disposed proximate the second end of the gas channel to nebulize the expelled liquid when the expelled liquid strikes the impactor; and
an overflow wall disposed between the reservoir and the exit port to deter the passage of liquid through the exit port.

22. A nebulizer for delivering a mist of liquid, comprising:
a housing having an exit port for delivering a mist of liquid, wherein the housing comprises an upper housing portion and a lower housing portion that may be joined together to form an enclosure, each housing portion having a sidewall with an interior wall surface;
a reservoir disposed internally to the housing for containing a liquid to be nebulized;
a nebulizer tube having:
a gas channel having a first end for receiving a compressed gas and a second end for expelling compressed gas and nebulized liquid, and
a liquid feed channel having a first end in fluid communication with the reservoir for receiving the liquid from the reservoir and having a second end in fluid communication with the gas channel, whereby application of compressed gas to the first end of the gas channel creates a siphon in the liquid feed channel to draw liquid into the feed channel and to expel the liquid and compressed gas from the second end of the gas channel;
an impactor disposed proximate the second end of the gas channel to nebulize the expelled liquid when the expelled liquid strikes the impactor; and
a curtain wall that is positioned to provide a protective curtain at the point of joinder between the upper and lower housing portions to deter passage of liquid into the point of joinder.

23. The nebulizer according to claim 22, wherein the upper housing supports the curtain wall so that the curtain wall extends downward into the lower housing portion past the point of joinder between the upper and lower housing portions.

24. The nebulizer according to claim 22, wherein the curtain wall is configured to redirect any accumulation of liquid on the upper surface of the upper housing portion downward into the reservoir.

25. The nebulizer according to any one of claims 22, wherein the curtain wall is positioned relative to the impactor to permit the mist to travel by the curtain wall around the impactor to the exit port.

26. The nebulizer according to any one of claims 22, wherein the curtain wall comprises a curved surface.

27. A nebulizer for delivering a mist of liquid, comprising:
a housing having an exit port for delivering a mist of liquid;
a reservoir disposed internally to the housing for containing a liquid to be nebulized;
a nebulizer tube having:
a gas channel having a first end for receiving a compressed gas and a second end for expelling compressed gas and nebulized liquid, wherein the gas channel comprises a convergent channel that has a cross-sectional dimension that decreases from the first end toward the second end, and
a liquid feed channel having a first end in fluid communication with the reservoir for receiving the liquid from the reservoir and having a second end in fluid communication with the gas channel, and wherein the gas channel has a minimal cross-sectional dimension proximate the second end of the liquid feed channel, whereby application of compressed gas to the first end of the gas channel creates a siphon in the liquid feed channel to draw liquid into the feed channel and to expel the liquid and compressed gas from the second end of the gas channel; and
an impactor disposed proximate the second end of the gas channel to nebulize the expelled liquid when the expelled liquid strikes the impactor.

28. A nebulizer for delivering a mist of liquid, comprising:
a housing having an exit port for delivering a mist of liquid;
a reservoir disposed internally to the housing for containing a liquid to be nebulized, wherein the reservoir comprises depression into which the liquid may pool;
a nebulizer tube having:
a gas channel having a first end for receiving a compressed gas and a second end for expelling compressed gas and nebulized liquid, and
a liquid feed channel having a first end in fluid communication with the reservoir for receiving the liquid from the reservoir and having a second end in fluid communication with the gas channel, and wherein the first end of the liquid feed channel is disposed proximate the deepest portion of the reservoir, whereby application of compressed gas to the first end of the gas channel creates a siphon in the liquid feed channel to draw liquid into the feed channel and to expel the liquid and compressed gas from the second end of the gas channel; and
an impactor disposed proximate the second end of the gas channel to nebulize the expelled liquid when the expelled liquid strikes the impactor.
